(19) United States
(12) Patent Application Publication
(10) Pub. No.: US 2006/0142206 A1
(43) Pub. Date: Jun. 29, 2006

(54) Novel difluorinated gem compounds, preparation methods thereof and applications of same

(75) Inventors: Jean-Charles Quirion, Bourg Achard (FR); Xavier Panecoucke, Maromme (FR); Francois D'Hooge, Mont Saint Aignan (FR); Stephane Marcotte, Buchy (FR); Geraldine Castelot-Deliencourt-Godefroy, Maisons Alfort (FR); Philippe Jubault, Preaux (FR); Vanessa Gouge, Rouen (FR)

Correspondence Address: BROWDY AND NEIMARK, P.L.L.C.;624 NINTH STREET, NW, SUITE 300, WASHINGTON, DC, 20001-5303, US

(73) Assignee: Institut National des Sciences Appliquees, Mont Saint Aignan (FR)

(21) Appl. No.: 10/522,365

(22) PCT Filed: Jul. 23, 2003
(86) PCT No.: PCT/FR03/02330

§ 371 Date: Sep. 21, 2005

(30) Foreign Application Priority Data
Jul. 25, 2002 (FR) 0209627

(51) Int. Cl. A61K 31/70

(52) U.S. Cl. 514/23

(57) ABSTRACT

This invention relates to a mini-type electric vehicle, which is mainly used for individual person or traffic in a certain field. It includes frame, seat, front & rear wheel, driving device, battery, steering system and front wheel suspension device. A casing, where the battery is placed on, protrudes forward at the middle position of the front end of the frame; the suspension device for front wheel appears “Front convex & rear concave” shape and covers the front end of that casing, and pivots to the middle of the front end of the casing. Two front wheels are installed on the front wheel suspension device. The rear edge line of the two front wheels is located at the rear of the battery front edge; Steering system is connected to the front end of the frame and interlocks with the front wheel. Battery is installed at the front so as to balance the weight distribution, improve the driving safety, comfort and steering performance for electric vehicle.

The invention relates to a method for the synthesis of gem-difluorinated compounds. More specifically, but not exclusively, it applies to the preparation of glycoconjugated compounds and C-glycosides notably for making antitumoral, antiviral, hypoglycemic, anti-inflammatory agents or even for immunology, cosmetology and the preparation of glycopeptide analogs of antifreeze molecules.

In recent years, the number of investigations relating to fluorinated organic molecules has increased considerably. This enthusiasm is explained by the recognition of the impact of fluorine in the biological activity of the molecules. Indeed, physiological properties of bioactive compounds are changed with the introduction of fluorine and biochemists are eager for new methods for selectively introducing fluorine.

However, the main contributions as regards new important biological molecules have essentially been made in monofluorination and trifluorination.

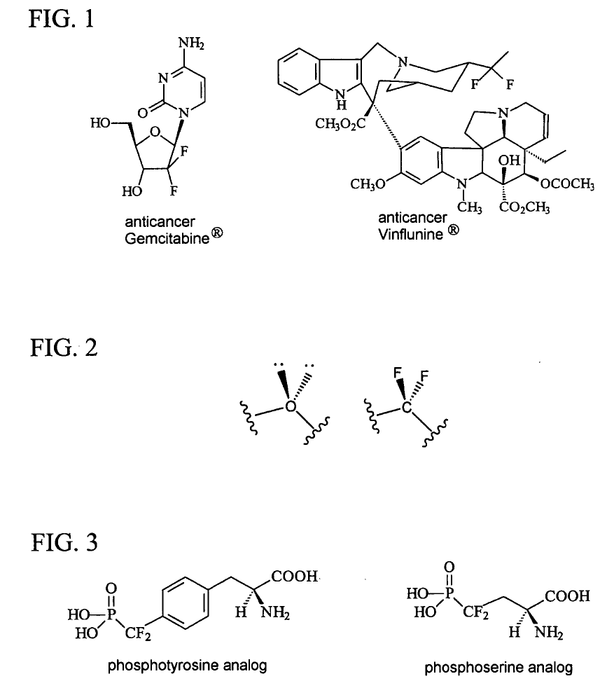

The introduction of the difluoromethylene CF₂ group has nevertheless shown significant importance in compounds such as Gemcitabine® (Gemzar, Lilly) and Vinflunine® (Pierre Fabre) which are presently undergoing clinical trials as antitumoral agents (FIG. 1).

This interest for selective fluorination of biological compounds is related to the very nature of the fluorine atom: its electronegativity (the most electronegative element), the C-F binding energy (484 kJ.mol⁻¹; C-C: 348 kJ.mol⁻¹).

As a replacement for oxygen, the difluoromethylene CF₂ group has proved to be a particularly attractive candidate:
Electronegativity of oxygen (3.5) is rather close to that of the CF₂ group (3.3), on the one hand;
on the other hand, during preliminary investigations carried out in 1984 with replacement of oxygen of a phosphate analog in adenosine diphosphate (ADP) type structures, it was shown that CF₂ was a tetrahedral equivalent of oxygen by the spatial arrangement of both fluorines, as illustrated in FIG. 2.

Moreover, as the electronegativities are very close, electronic effects due to the replacement are minimized.

Hence, analogs of phosphotyrosine and phosphoserine illustrated in FIG. 3 have been recently synthetized.

These compounds are inhibitors of phosphatase enzymes which are involved in the transduction of intracellular signals.

Moreover, syntheses of glycoconjugated compound analogs are carefully under investigation. These are compounds formed by conjugation between a sugar and another compound (aglycone) such as an amino acid (glycoprotein, glycopeptide), a lipid (glycolipid), a steroid or a triterpene, an alkaloid, a ketone . . .

Indeed, the latter, with i.a. glycoprotein and glycolipid which are constituents of cellular membranes, are compounds widely involved in many biochemical processes such as intercellular recognition or cell growth control. For this reason, glycoconjugated compounds are a considerable therapeutic wager and find applications as antitumoral or antiviral agents.

Now, these compounds owing to the presence of an osidic bond (a bond involving oxygen said to be in an anomeric position) are fragile relatively to several enzymatic systems including protease enzymes and hydrolase enzymes.

In order to have the components retain their biological properties, replacement of the oxygen of the osidic bond is therefore of interest, so that this bond is no longer degraded by an enzymatic process.

Analogs where oxygen is replaced with CH₂, have been synthetized, but, in spite of an increase in stability and sterical hindrance similar to that of oxygen, the CH₂ group has not proved to be a good mimic of biological properties of the initial compound.

Other classes of compounds where oxygen is replaced with nitrogen or sulphur, and more recently with a difluoromethylene group are being investigated in order to impart increased stability to glycoconjugated compounds in a biological medium.

This O/CF₂ transposition seems particularly suitable for mimicking oxygen on the electronic level; both fluorine atoms playing the role of both free doublets of oxygen (FIG. 2).

Several teams are investigating access to C-glycosides (compounds where anomeric oxygen is replaced with a carbon) but no effective method applicable to the large range of sugars encountered in glycoconjugated compounds (D-glucose, D-galactose, D-galactosamine, D-glucosamine . . . ) has been reported to this day.

More specifically, the object of the invention is therefore to remedy such drawbacks.

For this purpose, it proposes a gem-difluorinated C-glycoside compound of general formula I: 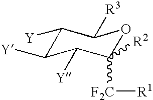
wherein

R¹ is a group comprising an alkyl chain substituted with at least one amine, amide, or acid function,

R² is a hydrogen atom H or a free or protected alcohol function,

R³ is notably an H, CH₃, CH₂OH, CH₂-OGP group wherein GP is a protective group such as an alkyl, benzyl (Bn), trimethylsilyl (TMS), tert-butyl-dimethylsilyl (TBDMS), tert-butyldiphenylsilyl (TBDPS), acetate (Ac) . . . ,

Y, Y′, Y″ are independent groups

wherein Y, Y′, Y″=H, OR, N₃, NR′R″, SR′″. . .
with R=H, Bn, Ac, TMS, TBDMS, TBDPS, . . . ,
R′, R″=H, alkyl, allyl, Bn, tosylate (Ts), C(═O)-alkyl, C(═O)-Bn, . . . ,
R′″=H, alkyl, Ac.

In addition, this compound of general formula I may be prepared by a reaction between a lactone with general formula II:

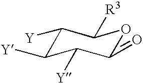

wherein R³ is notably a H, CH₃, CH₂-OGP wherein GP is a protective group such as an alkyl, benzyl (Bn), trimethylsily (TMS), tert-butyldimethylsilyl (TBDMS), tert-butyldiphenylsilyl (TBDPS), acetate (Ac) . . . ,

Y, Y′, Y″ are independent groups

wherein Y, Y′, Y″=H, OR, N₃, NR′R″, SR″′
with R=H, Bn, Ac, TMS, TBDMS, TBDPS, . . . ,
R′, R″=H, alkyl, allyl, Bn, tosylate (Ts), C(=O)-alkyl, C(=O)-Bn, . . . ,
R′″=H, alkyl, Ac.

and at least one halogenated derivative with general formula XCF₂CO₂R⁸, wherein X is a halogen, in the presence of zinc, or of a lanthanide derivative and R⁸=alkyl, aryl . . .

Said lanthanide derivative may for example be samarium diiodide SmI₂.

According to an alternative, said method may use zinc associated with titanocene.

Deoxygenation for passing from a compound of formula I wherein R²=OH to a compound with formula I wherein R²=H, may for example be either achieved by direct or radical reduction or even via acetate, tosylate, xanthate, oxalate derivatives, followed by radical reduction.

According to one alternative embodiment, more specifically, the gem-difluorinated compounds may have general formula III: 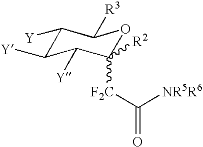
wherein R⁵ and R⁶=H or a group either functionalized or not such as a functionalized carbon chain bearing i.a. an amine, amino acid, aminoester function, a peptide chain, a protein, a carbohydrate, a steroid, or a triterpene, an alkaloid, a lignane, or compounds of pharmacological interest . . .

According to another alternative, the gem-difluorinated compounds may more specifically have general formulae IVa and IVb: 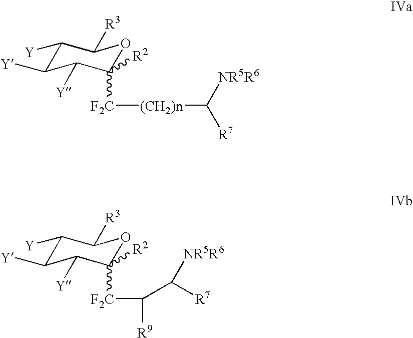
wherein R⁵, R⁶, R⁷ and R⁹=H or a group either functionalized or not, such as a functionalized carbon chain bearing i.a. an amine, amino acid, aminoester function, a peptide chain, a protein, a carbohydrate, a steroid, or a triterpene, an alkaloid, a lignane, or compounds of pharmacological interest.

One of the intermediate compounds obtained for obtaining compound of formula I may be a compound of general formula V including an ester function: 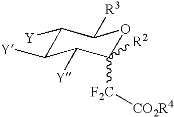
wherein R⁴ may be a group such as an alkyl, aryl, allyl group, this group either being functionalized or not.

This ester function —CO₂R⁴ may be saponified in order to obtain the acid of formula VI:

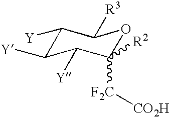

This ester function —CO₂R⁴ may also be reduced to an alcohol function, for example with sodium tetraborohydride (NaBH₄) or lithium aluminium tetrahydride (LiAlH₄) in order to give C-glycoside compounds of general formula VII:

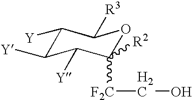

These compounds of general formula VII may themselves be oxidized into aldehydes by different methods such as the methods of Swern, Dess-Martin in order to obtain compounds of general formula VIII:

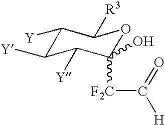

Compounds VIII are also accessible from esters V via the thioester and reduction.

Compound VIII may be obtained in the hemiacetalic form.

Non-osidic compounds of formula I, wherein R¹═CH₂—OH, may also be oxidized into aldehydes with either of the aforementioned methods.

In addition, according to another alternative, compounds of formula I wherein R¹═COOH may be used in a Ugi reaction with an amine, an aldehyde and an isonitrile for obtaining compounds of formula III wherein R¹═—C(═O)—NR⁵R⁶.

According to a last alternative embodiment, compounds of general formula I may be obtained by coupling a sugar derivative with an amine, for example an amino acid or a peptide.

Finally, the CF₂ group is particularly resistant to biochemical degradation processes and it therefore allows synthesis of non-hydrolyzable structures.

Compounds of general formulae I-VIII as well as their possible derivatives and pharmaceutically acceptable mineral or organic acid addition salts may for example exist as tablets, capsules, dragees, oral solutions or suspensions, emulsions, suppositories. In addition to pharmaceutically acceptable and non-toxic, inert excipients such as distilled water, glucose, starch lactose, talc, vegetable oils, ethylene glycol . . . , the thereby obtained compositions may also contain preservatives.

Other active ingredients may be added into these compositions.

The amount of compounds according to the invention and other possible active ingredients in such compositions may vary according to applications, the age, and the weight of the patient.

Examples for preparing the compounds according to the invention will be described hereafter by way of non-limiting examples.

The encountered acronyms are thereby defined:

eq.: equivalent

g: gram

Hz: hertz

mg: milligram

MHz: megahertz

min.: minute

mL: milliliter

mmol: millimole

μmol: micromole

nmol: nanomole

The examples hereafter describe the preparation of gem-difluorinated glycoconjugated compounds of general formula I:

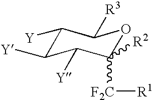

These compounds may be synthesized with different methods.

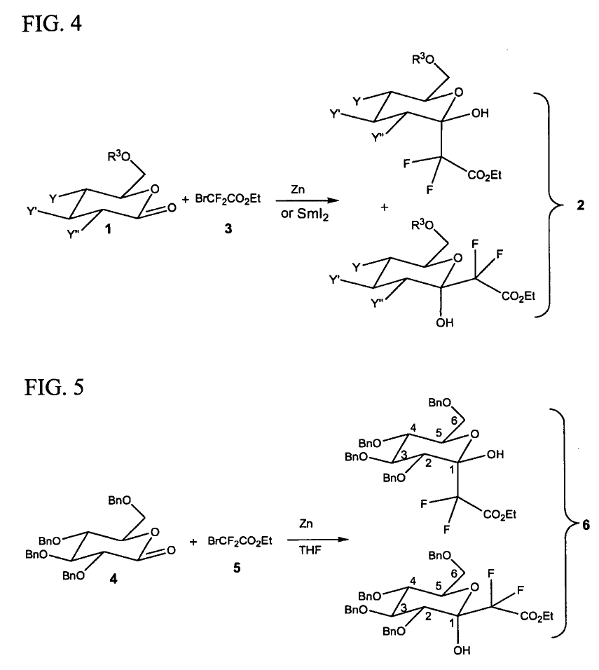

In order to reduce the number of steps during the synthesis of gem-difluorinated glycoconjugated compounds, lactones 1 were used as electrophilic substances (FIG. 4). Derivatives 2 were obtained from the lactones 1 by attack of ethyl bromodifluoroacetate 3 in the presence of zinc Zn or samarium diiodide SmI₂.

It should be noted that this method is general and may be applied to all the classes of differently substituted glucopyranoses (Y, Y′, Y″=OR, N₃, NR′R″, SR″ . . . ), the starting lactones being easily accessible in one or more steps from commercial products (for example in the glucose series, by oxidizing commercial products in one step).

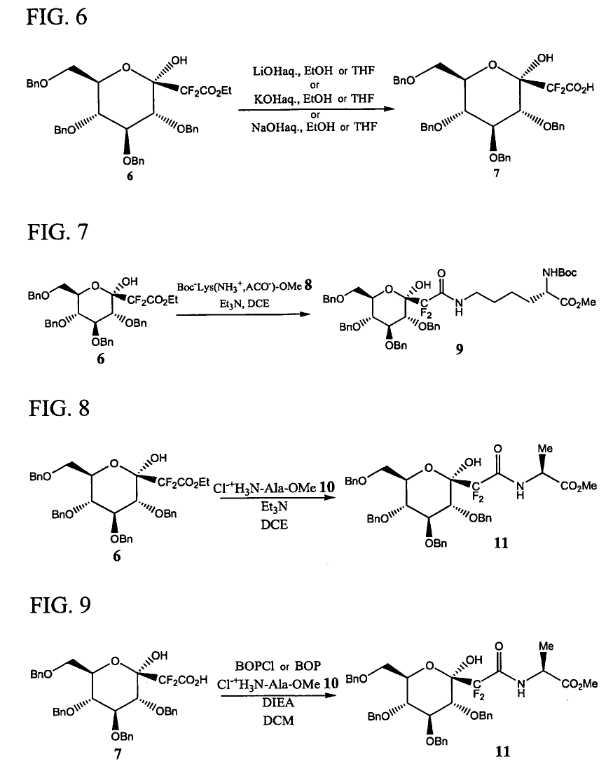

### SYNTHESIS OF INTERMEDIATE C-GLYCOSIDE COMPOUNDS 6 AND 7 (FIGS. 5 and 6):

In the example of FIG. 5, 0.82 g of activated zinc (Zn) (0.82 g, 12.5 mmol, 7 eq.) is introduced into a two-neck vial of 100 mL topped by a coolant and an inlet valve. The whole is put in vacuo and the zinc is heated with a heat pistol for about 5 min then the vacuum is released with an argon balloon.

15 mL of anhydrous tetrahydrofurane (THF) are added and the obtained solution is refluxed. The mixture, prepared under argon and consisting of the lactone 4 (0.960 g, 1.782 mmol, 1 eq.), of ethyl bromodifluoroacetate BrCF₂COOEt 5 (0.69 mL, 5.346 mmol, 3 eq.) and anhydrous tetrahydrofurane (15 mL) is introduced therein.

The assembly is left to reflux for 2 h 30 min (the reaction is followed by thin layer chromatography (TLC) with a (3:7) ethyl acetate/cyclohexane mixture as an eluent), then 30 mL of hydrochloric acid of concentration 1N and dichloromethane are added to the solution.

The phases are separated and extraction is achieved with dichloromethane (3×10 mL of dichloromethane are successively added to the aqueous phase and extracted) the organic phases are collected, dried on anhydrous magnesium sulfate (MgSO₄), filtered and concentrated on the evaporator in vacuo.

Separation is achieved by chromatography on a silica column with a cyclohexane/ethyl acetate mixture as eluent in proportions of nine for one. After concentration of the collected fractions, product 6 exists as a yellowish oil with a 89% yield by weight as a single diastereoisomer.

Compound 6 is obtained as a separable mixture of both diastereoisomers ((2:1) mixture) with a 62% yield by weight if samarium diiodide is used instead of zinc.

Characteristics of the devices used for performing the analyses of all the compounds described in the present application are indicated below:

¹H, ¹³C, ¹⁹F NMR spectra were recorded on BRUKER DPX 300 and DPX 600 spectrometers. In ¹H and ¹³C NMR, tetramethylsilane is used as an internal standard. In ¹⁹F-NMR, the external standard is fluorotrichloromethane (CFCl₃). Chemical shifts are expressed in parts per million (ppm), the coupling constants J in Hertz (Hz).

The following abbreviations were used:

s for singlet, b for a broad singlet, d for doublet, t for triplet, q for quadruplet, m for multiplet or massive, dd for doublet of doublet . . .

Infrared spectra were plotted on a PERKIN-ELMER PARAGON 500 FT-IR device in liquid film on sodium chloride crystal or in KBr tablet (for solids). The absorption frequencies are expressed in cm⁻¹.

Mass spectra were obtained on a JEOL AX 500 spectrophotometer with a FAB JEOL gun (Xe, 4 kV, 10 mA).

Separations by column chromatography were achieved under slight pressure by following the chromatographic techniques on Kieselgel 60 silica (230-400 mesh, Merck).

Follow-up is provided by chromatography on thin layers (TLC) with Kieselgel 60F-254-0.25 mm plates. The ratio of the migration distance of a compound on a given support over the migration distance of an eluent is called the front ratio (Rf).

The analyses performed for confirming the structure of the obtained product 6 are shown below:

Thin layer chromatography (TLC)

Rf=0.55, eluent: ethyl acetate/cyclohexane 3:7

NMR data:

¹⁹F-NMR (282 MHz; solvent: deuterated chloroform (CDCl₃)) −117.67, d, 2J_{F}. _{F}=256 Hz; −120.03, d, 2 J_{F-F}=256 Hz

¹H-NMR (300 MHz; solvent: deuterated chloroform (CDCl₃)) 1.19, t, ³J=7.14 Hz, 3H : CH₃(OEt); 3.52-3.70, m, 3H (H₅+2H₆); 3.90-3.95, m, 3H: H₂+H₃+H₄, 4.18, q, ³J=7.14 Hz, 2H: CH₂(OEt); 4.39-5.19, m, 8H: 4 CH₂(OBn); 7.14-7.24, m, 20H: 4×5 CH(Ph).

¹³C-NMR (75.5 MHz; solvent: deuterated chloroform (CDCl₃)): 14.29, CH₃(OEt); 63.89, CH₂(OEt); 68.68, CH₂(C₆); 73.06, CH; 73.82, 75.47, 75.67, 76.37: 4xCH₂(OBn); 77.83, CH; 78.62, CH; 83.79, CH; 96.59, dd, ²J_{C-F}=28.17 Hz and ²J_{C-F}=26.44 Hz, —CF₂C(OH)O—; 112.79, dd, ¹J_{C-F}=263.6 Hz and ¹J_{C-F}=259.6 Hz, CF₂; 137-138 CH(Ph); 163.32, dd, ²J_{C-F}=31.6 Hz and 2J_{C-F}=31.0 Hz, CFCOOEt.

IR (cm⁻¹)

4059.6, 3478.5, 3089.5, 3064.3, 3031.6, 2923.7, 2852.0, 2257.3, 2925.7, 1875.4, 1769.3, 1663.6, 1605.9, 1586.4, 1497.3, 1454.0, 1396.7, 1372.1, 1315.6, 1087.7, 1027.9, 910.6, 856.8, 802.1, 736.7, 698.1, 648.9, 605.5, 540.9, 462.7.

Mass spectrometry: FAB+ (Xe, 4 kV, 3-nitrobenzylalcohol matrix) 686(2%)=(M+Na)+, 663(4%)=M+, 661(6%), 572(3%)=(M−Bn)+, 554(3%)=(M−Bn−H₂O)+, 463(4%), 391(12%), 307(14%), 289(12%), 271(16%), 181(96%), 154(100%), 136(84%), 107(50%), 91(100%), 81(46%), 69(40%), 55(76%), 43(64%), 29(20%)

Deoxygenation to have access to derivatives 7 may then be performed through different routes (direct or radical reduction, via acetate, tosylate, xanthate derivatives . . . ).

Saponification may be performed quasi-quantitatively under different conditions whether with sodium, potassium or lithium hydroxides in an aqueous ethanol or THF solution (FIG. 6):

In a flask containing the ester 6 (0.5 g, 1.75 mmol 1 eq.) in tetrahydrofurane: (5 mL) or in ethanol (5 mL), an aqueous solution of lithine LiOH (2M, 0.75 mL, 2 eq.) or an aqueous caustic soda solution NaOH (0.07 g, 1.6 mmol) is added, then stirring is continued for twelve hours. The medium is evaporated when ethanol is used, then taken up with dichloromethane. The mixture is acidified with hydrochloric acid HCl 1M, then extracted several times with dichloromethane. The organic phases are collected, dried on MgSO₄, filtered and concentrated.

The obtained product is a colorless oil and the yield is quantitative.

NMR data:

¹⁹F-NMR (CDCl₃, 282.5 MHz)

−117.4, d (²J_{F-F}=258 Hz); −119.1, d (²J_{F-F}=258 Hz).

¹H-NMR (CDCl₃, 300 MHz):

3.40-3.60, m, 3H, H5 and H6; 3.90-4.00, m, 3H, H2, H3 and H4; 4.38-4.79, m,

8 H, 4 CH₂(OBn); 7.05-7.22, m, 20 H, H ar.

¹³C-NMR (CDCl₃, 75.5 MHz)

68.6 (C6); 72.2 (C5); 73.5, 75.5, 75.9, 76.4 (4 CH₂(OBn)); 77.7, 78.5, 83.6 (C2; C3 and C4); 96.0, dd, ²J_{C-F}=27.0 Hz and ²J_{C-F}=28.7 Hz , —CF₂Cl(OH)O—; 112.4 , dd, ¹J_{C-F}=260.3 Hz and ¹J_{C-F}=259.2 Hz, CF₂ 128.1, 128.2, 128.4, 128.8, 128.9, 129.0 (ar C.); 137.2, 137.7, 137.9, 138.6 (ar. C, quat) 163.6, dd, ²J_{C-F}=30.5 Hz and ²J_{C-F}=32.8 Hz, CF₂COOH.

Synthesis of a Difluorinated Gem-compound From Compounds 6 and 7

Reaction with amines

This reactivity enables access to very interesting compounds, analogs of glycopeptides.

Derivatives of compound 6 react with different primary or secondary amines leading to the corresponding amides. The amines used are aliphatic, benzyl or aromatic amines and amino acid derivatives such as lysine (FIG. 7):

In a flask under an inert atmosphere containing the starting product 6 (50 mg; 0.075 mmol; 1 eq.) in a solution and Boc-lysine-OMe acetate 8 (48 mg; 0.15 mmol; 2 eq.) in dichloroethane DCE (3 ml), triethylamine Et³N (53 μl; 0.375 mmol; 5 eq.) is added. The mixture is refluxed for forty eight hours and then the solvent is evaporated.

Purification of the crude product is achieved by chromatography on a silica column with a cyclohexane/ethyl acetate mixture as an eluent in proportions of seven for three.

After concentration, product 9 exists as a light yellow solid with a 84% yield by weight.

NMR data:

¹⁹F-NMR (CDCl₃, 282.5 MHz)

−117.4, d, (²J_{F-F}=259 Hz); −121.9, d, (²J_{F-F}=259 Hz).

¹H-NMR (CDCl₃, 300 MHz)

1.18-1.60, m, 15 H, (CH₃)₃C and (CH₂)₃; 3.06-3.19, m, 2 H, CH₂N; 3.52-3.69, m, 6 H, H5; H6 and CO₂CH₃; 3.84-4.18, m, 4 H, H2; H3; H4 and CHN; 4.36-4.85, m, 8 H, 4 CH₂Bn; 5.01, d, J=8.3 Hz, 1 H, NHBoc; 6.60, m, 1 H, NH; 7.10-7.23, m, 20 H, H ar.

¹³C-NMR (CDCl₃, 75.5 MHz) 22.7, 28.8 ((CH₂)₂); 28.9 ((CH₃)₃C); 32.5 (CH₂); 39.6 (CH₂N); 52.7 (CO₂CH₃); 53.6 (CHN); 68.7 (C6); 73.6, 75.3, 75.8, 76.4 (4 CH₂Bn); 72.1, 77.9, 78.6, 83.6 (C2, C3, C4 and C5); 96.1, dd, ²J_{C-F}=27.4 Hz (CF₂CO(OH)); 112.5, dd, ¹J_{C-F}=261.7 Hz (CF₂); 127.6, 127.7, 127.8, 128.3, 128.4, 128.5 (ar. C) , 137.5, 137.9, 138.0, 138.3 (ar. C quat.); 155.6 (CO₂Me); 163.7, dd, ²J_{C-F}=27.4 Hz (CF₂CONH); 173.3 (NHCO₂tBu).

A glycosylated derivative of alanine may be obtained from compound 6 (FIG. 8) or from compound 7 (FIG. 9) according to three different procedures:

The first procedure A is identical with that used for compound 9 derived from lysine. The weight yield for compound 11 is 30% (FIG. 8).

The second procedure B (FIG. 9) is the following:

BOP (benzotriazol-1-yloxy-tris(dimethylamino)-phosphonium hexafluorophosphate) (35 mg; 7.87*10⁻³ MMol; 1 eq.) and diisopropylethylamine DIEA (28 μL; 0.016 mmol; 2 eq.) are introduced into a flask under an inert atmosphere containing the acid 7 (50 mg; 7.87*10⁻³ mmol; 1 eq.) in dichloromethane DCM (2 mL). The reaction medium is stirred for one hour and then a solution consisting of alanine 10 (11 mg; 7.87*10⁻³ mmol; 1 eq.) and DIEA (14 μL; 7.87*10⁻³ mmol; 1 eq.) in dichloromethane (2 mL) is added to the reaction. Stirring is continued for twenty four hours. The medium is then washed with a solution of hydrochloric acid HCl 1M. The organic phase is dried on magnesium sulfate, filtered and evaporated.

The crude product is then purified on a preparatory silica plate by using a cyclohexane/ethyl acetate mixture as eluent in proportions of seven to three.

Product 11 is obtained as white crystals with a 77% yield by weight.

The third procedure C (FIG. 9) is the following:

In a flask under an inert atmosphere containing the acid 7 (50 mg; 7.87*10⁻³ mmol; 1 eq.) in dichloromethane (2 mL), BOPCl (bis-(2-oxo-3-oxazolidinyl)phosphinic chloride) (40 mg; 7.87*10⁻³ mmol; 1 eq.) and diisopropylethylamine DIEA (28 μL; 0.016 mmol; 2 eq.) are introduced. Next stirring is continued for one hour before adding to the reaction a solution consisting of the alanine derivative 10 (22 mg; 0.016 mmole; 2 eq.) and diethylamine DIEA (44 μL; 0.023 mmole; 3 eq.) in dichloromethane (2 mL). Stirring is continued for twenty four hours then the medium is washed with a HCl 1M solution. The organic phase is dried on magnesium sulfate, filtered and evaporated.

The crude product is then purified on a preparatory silica plate by using a cyclohexane/ethyl acetate mixture as an eluent, in proportions of seven to three.

Product 11 is obtained as white crystals with a 44% yield by weight.

NMR data:

¹⁹F-NMR (CDCl₃, 282.5 MHz)

−118.0, d, (²J_{F-F}=259 Hz); −122.2, d, (²J_{F-F}=259 Hz).

¹H-NMR (CDCl₃, 300 MHz)

1.26, d, ³J=7.2 Hz, 3 H, CH₃; 3.50-3.66, m, 3 H, H5 and H6; 3.63, S, 3 H, CO₂CH₃; 3.89-3.96, m, 3 H, H2, H3 and H4; 4.40-4.81, m, 10 H, NH; CHN and 4 CH₂Bn; 7.11-7.21, m, 20 H, ar. H. ¹³C-NMR (CDCl₃, 75.5 MHz) 16.7 (CH₃); 47.2 (CHN); 51.7 (CO₂CH₃); 67.3 (C6); 72.3, 73.9, 74.3, 75.0 (4 CH₂Bn); 70.9, 76.2, 77.1, 82.2 (C2, C3, C4 and C5); 126.6-127.4, m (ar. C); 136.5, 136.9, 137.0, 137.4 (ar. C, quat.); 171.0 (CO₂Me).

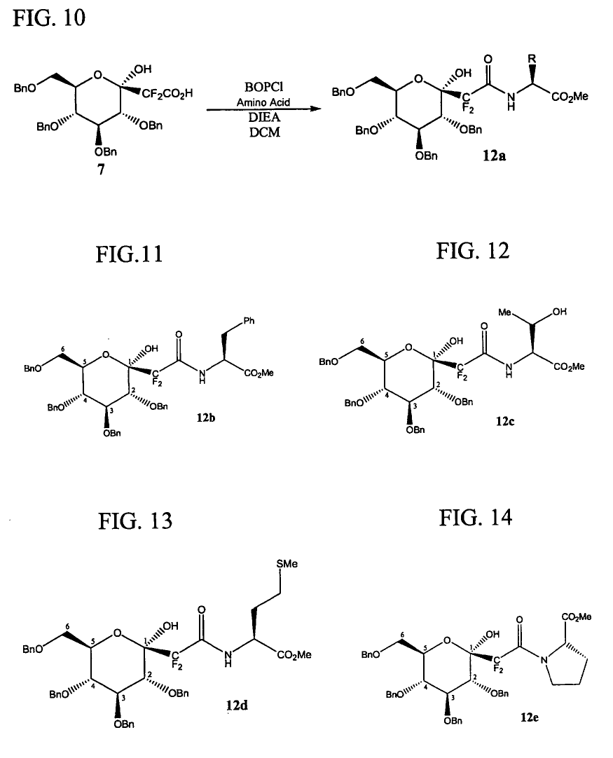

Coupling reactions with the following amino acids such as phenylalanine, threonine, methionine, proline as well as with a dipeptide were achieved by using BOPCl as coupling agent, i.e., by following the same method as procedure C upon coupling with alanine (FIG. 10).

Product 112b is obtained as white crystals with a 42% yield by weight (FIG. 11).

NMR data:

¹⁹F-NMR (CDCl₃, 282.5 MHz)

−117.7, d, (²J_{F-F}=261 Hz); −121.6, d, (²J_{F-F}=261 Hz).

¹H-NMR (CDCl₃, 300 MHz)

3.07, m, 2 H, CH₂Ph; 3.44-3.67, m, 3 H, H5 and H6; 3.57, S, 3 H, CO₂CH₃; 3.91-3.98, m, 3 H, H2, H3 and H4; 4.25-4.85, m, 10 H, NH, CHN and 4

CH₂Bn; 7.00-7.14, 25 H, ar. H

¹³C-NMR (CDCl₃, 75.5 MHz)

37.5 (CH₂Ph); 52.4 (CO₂CH₃); 53.1 (CHN); 68.3 (C6); 73.2, 75.0, 75.3, 76.0 (4 CH₂Bn); 72.0, 77.0, 78.2, 83.2 (C2, C3, C4 and C5); 127.3-129.3, m (ar. C); 135.0, 137.5, 137.9, 138.0, 138.4 (ar. C quat.); 170.3 (CO₂Me).

Product 12c is obtained as white crystals with a 28% yield by weight (FIG. 12).

NMR data:

¹⁹F-NMR (CDCl₃, 282.5 MHz)

−118.3, d, (²J_{F-F}=257 Hz); −121.2, d, (²J_{F-F}=257 Hz).

¹H-NMR (CDCl₃, 300 MHz)

1.12, d, ³J=6.4 Hz, 3H, CH₃; 3.48-3.64, m, 3H, H5 and H6; 3.7, s, 3H, CO₂CH₃; 3.89-4.00, m, 3H, H2, H3 and H4; 4.22-4.82, m, 11H, NH; CHN, CHOH and 4 CH₂Bn; 7.0-7.24, m, 20H, ar. H

¹³C-NMR (CDCl₃, 75.5 MHz)

20.5 (CH₃); 53.2 (CO₂CH₃); 57.8 (CHN); 68.6 (CHOH); 68.7 (C6); 73.5, 75.4, 75.8, 76.4 (4 CH₂Bn); 72.2, 77.2, 78.4, 83.6 (C2, C3, C4 and C5); 128.1-128.9 m (ar. C); 137.8, 137.9, 138.1, 138.7 (ar. C quat.); 170.5 (CO₂Me).

Product 12d is obtained with a 36% yield by weight (FIG. 13).

NMR data:

¹⁹F-NMR (CDCl₃, 282.5 MHz)

−117.4, d, (²J_{F-F}=260 Hz), −121.7, d, (²J_{F-F}=260 Hz).

¹H-NMR (CDCl₃, 300 MHz)

1.89-1.99, m, 2H, CH₂; 2.09, s, 3H, SCH₃; 2.46, t, ³J=7.0 Hz, 2H, CH₂S; 3.58-3.77, m, 3H, H5 and H6; 3.68, s, 3H, CO₂CH₃; 3.96-4.03, m, 3H, H2, H3 and H4; 4.43-4.88, m, 1OH, NH; CHN and 4 CH₂Bn; 7.14-7.30, m, 20H, ar. H. ¹³C-NMR (CDCl₃, 75.5 MHz)

15.7 (CH₂); 29.9 (SCH₃); 31.6 (CH₂S); 51.8 (CO₂CH₃); 53.2 (CHN); 68.6 (C6); 73.6, 75.4, 75.8, 76.4 (4 CH₂Bn); 72.4, 77.4, 78.5, 85.6 (C2, C3, C4 and C5); 128.1-128.9 m (ar. C); 137.9, 138.3, 138.5, 138.8 (ar. C quat.); 171.5 (CO₂Me).

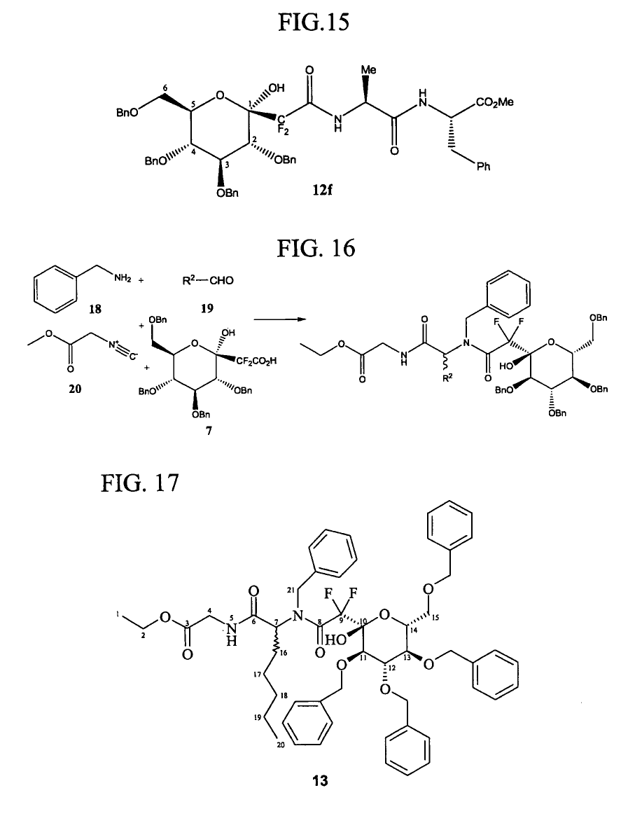

Product 12e is obtained as white crystals with a 32% yield by weight (FIG. 14).

NMR data:

¹⁹F-NMR (CDCl₃, 282.5 MHz)

−112.6, d, (²J_{F-F}=267 Hz); −113.7, d, (²J_{F-F}=261 Hz); −117.2 d (²J_{F-F}=261 Hz); −117.3, d, (²J_{F-F}=267 Hz).

¹H-NMR (CDCl₃, 300 MHz)

1.52-1.89, m, 4H, (CH₂)₂; 3.5-3.63, m, 3H, H5 and H6; 3.67, s, 3H, CO₂CH₃; 3.82-4.06, m, 5H, CH₂N; H2; H3 and H4; 4.33-4.92, m, 9H, CHN and 4 CH₂Bn; 7.10-7.20, m, 20H, ar. H.

Product 12f is obtained as white crystals with a 17% yield by weight (FIG. 15).

NMR data:

¹⁹F-NMR (CDCl₃, 282.5 MHz)

−117.6, d, (²J_{F-F}=257 Hz); −122.4, d, (²J_{F-F}=257 Hz).

¹H-NMR (CDCl₃, 300 MHz)

1.35, d, ³J=7.2 Hz, 3 H, CH₃, 3.05, m, 2 H, CH₂Ph; 3.5-3.71, m, 3H, H5 and H6; 3.70, s, 3H, CO₂CH₃; 3.89-4.01, m, 3H, H2; H3 and H4; 4.26-4.89, m, 11H, NH, 2 CHN and 4 CH₂Bn; 6.05, m, 1 H, NH; 7.10-7.20, m, 25H, ar. H

Compound 7 may also be used in a UGI reaction with an amine such as benzylamine 18, an aldehyde 19 and an isonitrile such as ethyl isocyanate 20 for compounds 13-17.

This is a route for accessing the synthesis of therapeutical compounds (manno- and fucopeptides) which are inhibitors of the bond between selectine and the tetrasaccharide, sialyl Lewis^{x} (sLe^{x}).

Leucocytes play an important role in many inflammatory and immunological phenomena. In many of these phenomena, the first steps are interactions between endothelial cells and leucocytes flowing in the blood.

Investigation of molecules specific to the surface of the cells, involved in these interactions, has shown that leucocytes and endothelial cells have at their surface specific lectins called selectins. These are cellular adhesion molecules of the family of calcium-dependent molecules. sLe^{x} is one of the ligands involved in the bonding between selectins, thereby causing adhesion of the leucocytes onto the endothelial tissue leading to acute forms of diseases such as rheumatismal arthrosis, psoriasis, cancer.

Consequently, development of sLe^{x}-inhibiting small size molecules is an attractive therapeutical approach.

Synthesis of Compound 13 (FIG. 17):

All the reagents are diluted in dry methanol in order to obtain a concentration of 1M.

In a 25 mL flask, a hexanal solution 0.081 mL; 0.675 mmol) is placed with a benzylamine solution 18 (0.059 mL; 0.54 mmole) and the mixture is stirred under argon for two hours at room temperature.

Next, a solution of ethyl isocyanoacetate 20 (0.074 mL; 0.675 mmol) and a solution of gem-difluorinated D-glucose as an acid 7 (286 mg; 0.45 mmol) are added and the mixture is stirred under argon for twenty four hours at room temperature.

Methanol is then evaporated and purification of the product is achieved by chromatography on a silica column with a gradient of ethyl acetate/cyclohexane as eluent, ranging in proportions from 1:9 to 2:8.

TLC

Rf=0.18, eluent: ethyl acetate/cyclohexane (2:8).

NMR data:

¹⁹F-NMR (CDCl₃)

−104.39 (d, ²J_{F-F}=260.1 Hz); −104.85 (d, ²J_{F-F}=257.9 Hz); −108.61 (d, ²J_{F-F}F=255.8 Hz); −108.89 (d, ²J_{F-F}=254.7 Hz); −108.95 (d, ²J_{F-F}=260.1 Hz); −112.49 (d, ²J_{F-F}=255.8 Hz); −114.35 (d, ²J_{F-F}=254.7 Hz); −116.17 (d, ²J_{F-F}=257.9 Hz). ¹H-NMR (CDCl₃)

0.69, t, 3H, H₂0, ³J_{H19-H20}=6.9 Hz; 0.90-1.10, m, 6H , 1.15, t, 5H, H₁, ³J_{H1-H2}=7.1 Hz; 3.41-3.74, m, 4H; 3.78-3.99, m, 4H; 4.07, q, 2H, H₂, ³J_{H1-H2}=7.1 Hz; 4.36-4.55, m, 4H; 4.61-6.97, m, 8H; 6.76, t, 0.7H, H₅, ³J_{H4-H5}=5.5 Hz; 6.82, t, 0.3H, H₅ rotamer, ³J_{H4-H5}=5.3 Hz; 7.00-7.26, m, 25H, H_{Ph}.

Mass spectrometry: (direct introduction, FAB+):

M+Na=959.6

M+K=975.7

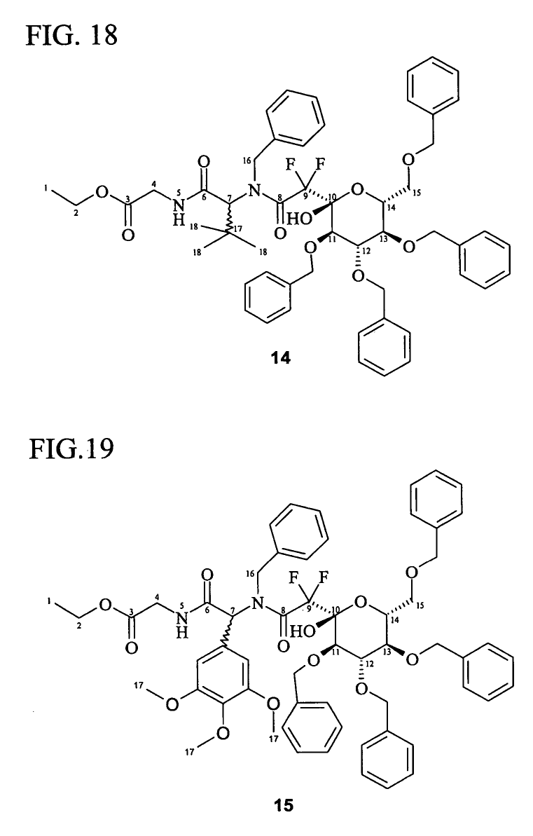

Synthesis of compound 14 (FIG. 18):

All the reagents are diluted in dry methanol in order to obtain a concentration of 1M.

A solution of trimethylacetaldehyde (0.073 mL; 0.675 mmol) is placed in a 25 mL flask, with a solution of benzylamine 18 (0.059 mL; 0.54 mmol) and the mixture is stirred under argon for two hours at room temperature.

Next, a solution of ethyl isocyanoacetate 20 (0.074 mL; 0.675 mmol) and a solution of gem-difluorinated D-glucose as an acid 7 (286 mg; 0.45 mmol) are added and the mixture is stirred under argon for twenty four hours at room temperature.

The methanol is evaporated and purification of the product is achieved by chromatography on a silica column with an ethyl acetate/cyclohexane gradient as eluent, in proportions from 1:9 to 3:7.

The obtained product is a yellow oil in the form of two diastereoisomers which are separated.

Analyses of the 1^{st} Diastereoisomer 14a

TLC

Rf=0.70, eluent: ethyl acetate/cyclohexane (4:6).

NMR data:

¹⁹F-NMR (CDCl₃):

−105.31 (d, ²J_{F-F}=267.0 Hz); −106.69 (d, ²J_{F-F}=267.0 Hz).

¹H-NMR (CDCl₃)

0.99, s, 9H, H₁₈; 1.16, t, 3H, H₁, ³J_{H1-H2}=6.9 Hz; 3.39-3.65, m, 4H; 3.90, dd, 2H, J=8.9 Hz; 4.00-4.15, q, 3H, H₂, ³J_{H1-H2}=6.9 Hz; 4.37, d, 1 H, J=11.7 Hz; 4.49, t, 2H, J=10.7 Hz; 4.69-4.97, m, 7H; 5.53, s, 1 H, H₇; 6.49, m, 1 H, H₅; 7.08-7.27, m, 25H, H_{Ph}.

Mass Spectrometry: (Direct Introduction, FAB+):

M+Na=945.4

Analyses of the 2nd Diastereoisomer 14b

TLC

Rf=0.65, eluent: ethyl acetate/cyclohexane (4:6).

NMR data:

¹⁹F-NMR (CDCl₃):

−107.15 (d, ²J_{F-F}=255.7 Hz).

¹H-NMR (CDCl₃)

1.02, s, 9H, H₁₈; 1.16, t, 3H, H₁, ³J_{H1-H2}=7.0 Hz; 3.52-4.00, m, 9H; 4.09, q, 2H, H₂, ³J_{H1-H2}=7.0 Hz; 4.33-4.86, m, 8H; 4.97, dd, 2H, H₁₆, H₁₆, ²J_{H16-H16}=17.3 Hz; 5.33, s, 1 H, H7; 6.49, m, 1 H, H₅; 6.98-7.27, m, 25H, H_{Ph},.

Mass spectrometry: (MALDI+):

M+Na=945.4

Synthesis of Compound 15 (FIG. 19):

All the reagents are diluted in dry methanol in order to obtain a concentration of 1M.

A solution of 3,4,5-trimethoxybenzaldehyde 22 (0.132 g; 0.675 mmol) is placed in a 25 mL flask, with a benzylamine solution 19 (0.059 mL; 0.54 mmole) and the mixture is stirred under argon for two hours at room temperature.

Next, a solution of ethyl isocyanoacetate 20 (0.074 mL; 0.675 mmol) and a solution of gem-difluorinated D-glucose as an acid 7 (286 mg; 0.45 mmol) are added and the mixture is stirred under argon for twenty four hours at room temperature.

The methanol is evaporated and purification of the product is achieved by chromatography on a silica column with an ethyl acetate/cyclohexane gradient as eluent, ranging in proportions from 1:9 to 3:7.

The obtained product is a yellow oil in the form of two diastereoisomers 15a, 15b, which are separated.

Analyses of the 1^{st} diastereoisomer 15a

TLC

Rf=0.41, eluent: ethyl acetate/cyclohexane (4:6).

NMR data:

¹⁹F-NMR (CDCl₃):

−111.63, s.

¹H-NMR (CDCl₃):

1.18, t, 3H, H₁, ³J_{H1-H2}=7.2 Hz; 3.38, t, 1 H, J=6.6 Hz; 3.58, s, 9H, H₁₇ 3.65, s, 4H; 3.93-4.14, m, 7H; 4.40-4.53, m, 3H; 4.70-4.87, m, 3H; 4.86, dd, 2H, H₁₆, H₁₆, 2J_{H16-H16′}=16.9 Hz; 5.33, s, 1 H; 6.38, s, 1 H, H₇; 6.43, t, 1 H, H₅, ³J_{H4-H5}=4.5 Hz; 6.90-7.25, m, 27H, H_{Ph}.

Mass spectrometry: (direct introduction, FAB+):

M+Na=1055.7

Analyses of the 2nd diastereoisomer 15b

TLC

Rf=0.32, eluent: ethyl acetate/cyclohexane (4:6).

NMR data:

¹⁹F-NMR (CDCl₃)

−108.12 (d, ²J_{F-F}=251.9 Hz); −115.19 (d, ²J_{F-F}=251.9 Hz).

¹H-NMR (CDCl₃)

1.17, t, 3H, H₁, ³J_{H1-H2}=7.0 Hz; 3.32-3.41, m, 1 H; 3.65, s, 9H, H₁₇; 3.70, s, 3H; 3.78-3.98, m, 5H; 4.08, q, 4H, H₂, ³J_{H1-H2}=7.0 Hz; 4.32, s, 2H; 4.60, dd, 2H, J=10.54 Hz; 4.67, s, 2H; 4.87, s, 1 H; 5.09, s, 1 H; 6.30, t, 1 H, H₅, ³J_{H4-H5}=4.9 Hz; 6.52, s, 2H, H₇; 6.86-7.23, m, 271, H_{Ph}.

Mass spectrometry: (direct introduction, FAB+):

M+Na=1055.7

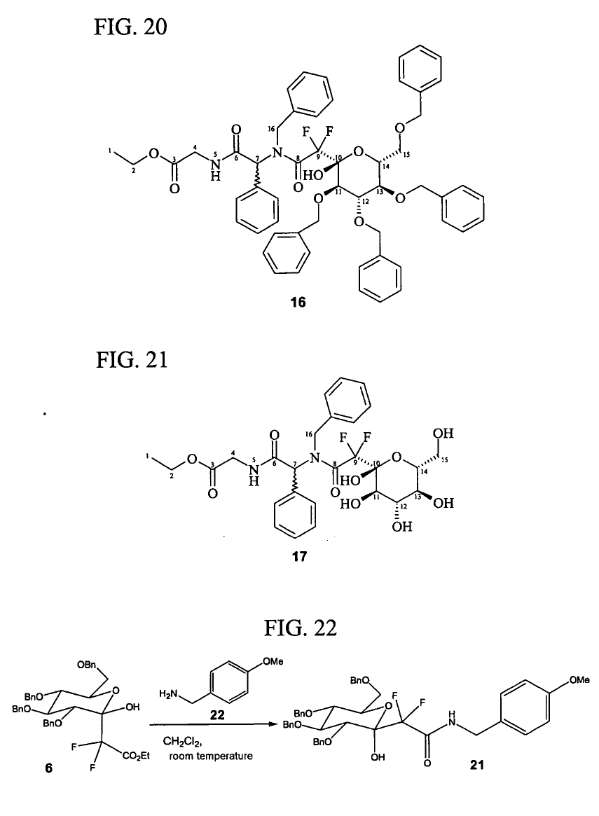

Synthesis of compound 16 (FIG. 20):

All the reagents are diluted in dry methanol in order to obtain a concentration of 1M.

A solution of benzaldehyde (0.059 mL; 0.675 mmol) is placed with the solution of benzylamine 18 (0.059 mL; 0.54 mmol) in a 25 mL flask, and the mixture is stirred under argon for two hours at room temperature.

Next, a solution of ethyl isocyanoacetate 20 (0.074 mL; 0.675 mmol) and a solution of difluorinated gem-D-glucose as an acid 7 (286 mg; 0.45 mmol) are added and the mixture is stirred under argon for twenty four hours at room temperature.

The methanol is evaporated and the purification of the product is achieved by chromatography on a silica column with an ethyl acetate/cyclohexane gradient as eluent ranging in proportions from 1:9 to 3:7.

The product is obtained in the form of two diastereomers 16a, 16b, which are separated.

Analyses of the 1^{st} diastereoisomer 16a

TLC

Rf=0.26, eluent: ethyl acetate/cyclohexane (3:7).

NMR data:

¹⁹F-NMR (CDCl₃):

−111.66, s, 2F.

¹H-NMR (CDCl₃):

1.15, t, 3H, H, ³J_{H1-H2}=7.0 Hz; 3.52-3.79, m, 3H; 3.83, dd, 1 H, J=4.5 Hz; 3.90-4.01, m, 4H, 4.07, q, 2H, H₂, J=7.0 Hz; 4.36-4.52, m, 4H; 4.68-4.82, m, 5H; 4.94, dd, 2H, H₁₆, ²J_{H16-H16}=15.8 Hz; 5.20, s, 1 H, H₇; 6.29, t, 1 H, H₅, ³J_{H4-H5}=4.5 Hz; 6.96-7.23, m, 30H, H_{Ph}.

¹³C-NMR (CDCl₃): 14.2, C₁; 41.6, C₄; 52.0, 61.6, C₂; 66.2, 68.5, 71.7, 73.5, 75.1, 75.4, 75.9, 77.5, 78.6, 83.5, 96.9, t, C₁₀, ²J_{C10-F}=27.6 Hz; 114.3, t, C₉, ¹J_{C-F}=262.9 Hz; 126.9, 127.2, 127.7, 127.8, 127.9, 128.0, 128.1, 128.2, 128.3, 128.4, 128.5, 128.5, 128.6, 128.8, 130.0, 133.0, 136.3, 137.8, 138.0, 128.6, 165.1, t, C₈, ²J_{C8-F}=26.4 Hz; 168.3; 169.7.

Mass spectrometry: (MALDI+):

M+Na=965.5

M+K=981.5

Analyses of the 2 diastereoisomer 16b

TLC

Rf=0.71, eluent: ethyl acetate/cyclohexane (5:5).

NMR data:

¹⁹F-NMR (CDCl₃)

−107.71 (d, ²J_{F-F}=253.1 Hz); −115.09 (d, ²J_{F-F}=253.1 Hz).

¹H-NMR (CDCl₃)

1.16, t, 3H, H₁, ³J_{H1-H2}=7.0 Hz, 3.35-3.40, m, 1 H; 3.51-3.70, m, 4H; 3.84-4.00, m, 5H; 4.08, q, 2H, H₂, ³J_{H1-H}₂=7.0 Hz; 4.23, s, 1 H; 4.62, dd, 2H, J=9.98 Hz; 4.67, s, 1 H; 4.81, d, 1 H, J=3.8 Hz; 4.98, s, 1 H; 5.08, d, 1 H, H₁₆ or H_{16′}, ²J_{H16-H16′}=18.0 Hz; 6.08, t, 1 H, H₅, ³J_{H4-H5}=4.9 Hz; 6.76-6.85, m, 1 H; 6.95-7.29, m, 30H, H_{Ph}.

Mass spectrometry: (MALDI+):

M+Na=965.4

M+K=981.3

Synthesis of the compound 17 (FIG. 21):

The first diastereoisomer of (2-{benzyl-[2,2-difluoro-2-(3(R),4(S)-tris-benzyloxy-6(R)-benzyloxy-methyl-2(R)-hydroxytetrahydro-pyran-2-yl)-acetyl)-amino}-2-phenylacetylamino)-acetic acid ethyl ester 16a (0.139 g; 0.147 mmol) is placed in a 25 mL flask with 6.6 mL of methanol and a dash of 10% palladium on charcoal (Pd/C) from a spatula. After having applied vacuum, a hydrogen balloon is set up and stirring is maintained overnight at room temperature.

The solution is filtered on celite, then evaporated in order to obtain product 17 as white crystals.

NMR data:

−¹⁹F-NMR (CD₃OD)

−108.37 (d, ²J_{F-F}=261.7 Hz); −109.29 (d, ²J_{F-F}=256.8 Hz), −111.04 (d, ²J_{F-F}=261.7 Hz); −115.44 (d, ²J_{F-F}=256.8 Hz); −120.50, s.

¹H-NMR (CD₃OD)

1.19, t, 3H, H₁, ³J_{H1-H2}=7.1 Hz; 3.39-3.52, m, 1 H; 3.59-3.98, m, 7H; 4.044.19, m, 2H; 4.28, dd, 1 H, ²J=17.7 Hz; 5.22, dd, 1 H, H₁₆, H₁₆, ²J_{H16-H16′}=17.7 Hz; 5.67, s, 1 H, H₇; 6.69-7.40, m, 10H, H_{Ph}.

Mass spectrometry: (direct introduction, FAB+):

M+Na=605.0

In the glucose series, preparation of the amide 21 is described (FIG. 22).

In a 50 mL flask under argon, ester 6 (0.193 g, 0.291 mmol, 1 eq.) is dissolved in anhydrous dichloromethane (5 mL). Para-methoxybenzylamine 22 (0.057 mL, 0.436 mmol, 1.5 eq.) is added and the mixture is left under stirring overnight. The solution is then evaporated in vacuo.

Purification is achieved by chromatography on a silica column with a cyclohexane/ethyl acetate as eluent in proportions of nine for one.

After concentration, product 21 exists as a white solid with a 56% yield by weight.

Analyses carried out for confirming the structure of the obtained product 21 are shown below:

TLC

Rf=0.52, eluent: ethyl acetate/cyclohexane (3:7).

NMR data:

¹⁹F-NMR (282 MHz; solvent: deuterated chloroform (CDCl₃)): −117.38, d, J_{F-F}=257 Hz; −121.90, d, J_{F-F}=257 Hz

¹H-NMR (300 MHz; solvent: deuterated chloroform (CDCl₃))

3.3-5, m, 16H (cycle+4xOBn); 3.66, s, 3H: CH₃ (OMe); 6.73, d, J=8.4 Hz, 2H: 2CH (PMB); 7.07, d, J=8.4 Hz, 2H: 2CH (PMB); 7.14-7.24, m, 20H: 4x5 CH (Ph).

¹³C-NMR (75.5 MHz; solvent: deuterated chloroform (CDCl₃)):

43.35, CH₂(PMB); 55.68, CH₃(OMe), 68.68, CH₂(C6); 73.06, CH; 73.82, 75.47, 75.67, 76.37: 4xCH₂(OBn); 77.83, CH; 78.62, CH; 83.79, CH; 96.59, dd, J_{C-F}=28.17 Hz and J_{C-F}=26.44 Hz, —CF₂CH(OH)O—; 112.79, dd, J_{C-F}=263.6 Hz and J_{C-F}=259.6 Hz, CF₂; 114.60, 2 CH(PMB); 137-138 CH(Ph+PMB); 159.71, C quat. (C-OMe PBM);

163.32, dd, J_{C-F}=31.6 Hz and J_{C-F}=31.0 Hz, CF₂CONH.

Reduction of the ester function

By transforming the ester function of difluoroacetylated C-glycosides into other functions, a wide range of glycoconjugates may be accessed. The reactivity of this α ester function of a difluoromethylene group and notably its reduction were investigated.

The ester function of compounds 2 (or 6) is reduced to an alcohol function by sodium tetraborohydride (NaBH4) or lithium aluminium tetrahydride (LiAlH₄) in order to obtain compound 23 (FIG. 22). The alcohol function of this compound is then oxidized into an aldehyde function in order to obtain compound 24 by different methods such as Swern's, Dess-Martin's methods . . .

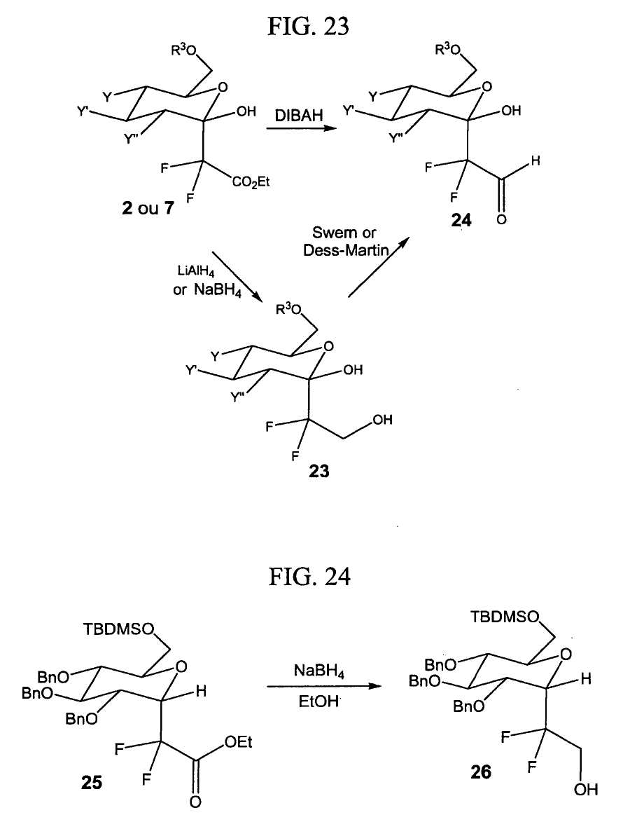

It should be noted that direct reduction of the alcohol into an aldehyde by diisobutylaluminium hydride (DIBAH) is possible on non-osidic compounds.
Reduction of an ester 25 into an alcohol 26 (FIG. 23).

The ester 25 (30 mg; 45 nmol; 1 eq.), sodium tetraborohydride NaBH₄ (5 mg; 134 nmol; 3 eq.) and 5 mL of ethanol (EtOH) are placed in 25 mL flask.

The solution is left under stirring at room temperature overnight and then dry evaporated in vacuo.

The white precipitate is redissolved in 10 ml of water and 10 ml of dichloromethane.

The phases are separated, the aqueous phase is extracted with dichloromethane (2×10 mL) and the organic phases are collected, dried on anhydrous magnesium sulphate and evaporated in vacuo to afford 24 mg of the alcohol 26 (38 nmol) with a 86% yield by weight.

Analyses carried out for confirming the structure of the obtained product 26 are shown below:

TLC

Rf=0.44, eluent: ethyl acetate/cyclohexane (8:2).

NMR data:

¹⁹F-NMR (282 MHz, solvent: deuterated chloroform (CDCl₃))

−110.68, dm, ²J_{F-F}=259.7 Hz, J_{F-H} not measurable; −117.8, dm, ²J_{F-F}=259.7 Hz, J_{F-H} not measurable

¹H-NMR (300 MHz, solvent: deuterated chloroform (CDCl₃))

0.00, s, 6H (2x CH₃ TBDMS); 0.84, s, 9H (3x CH₃ TBDMS);. 3.39-4.96, m, 15H; 7.23-7.33, m, 15H (3x 5CH Ph)

¹³C-NMR (75.5 MHz, solvent: deuterated chloroform (CDCl₃))-DEPT 135-5.04 and −5.09, 2CH₃(TBDMS), 26.25, 3 CH₃(TBDMS); 62.37, CH₂(C6); 64.16, CH₂, t, ²J_{C-F}=31 Hz (CF₂CH₂OH); 73.23 , 74.87 et 75.64, 3x CH₂ (OBn); 73.45, 74.80 , 79.52 and 84.81, 4x CH (C2 à C5); 78.15, CH, dd, ²J_{C-F}=26 and 29 Hz; 128.1-128.9, 3x 5 CH (OBn).

1. A gem-difluorinated compound of formula: 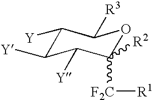
wherein
R¹ is a group comprising an alkyl chain substituted with at least one amine, amide, or acid function,
R² is a hydrogen atom H or a free or protected alcohol function,
R³ is notably an H, CH₃, CH₂OH, CH₂-OGP group wherein GP is a protective group such as an alkyl, benzyl (Bn), trimethylsilyl (TMS), tert-butyl-dimethylsilyl (TBDMS), tert-butyldiphenylsilyl (TBDPS), acetate (Ac) . . . ,
Y, Y′, Y″ are independent groups
wherein Y, Y′, Y″=H, OR, N₃, NR′R″, SR′″ . . .
with R=H, Bn, Ac, TMS, TBDMS, TBDPS, . . . ,
R′, R″=H, alkyl, allyl, Bn, tosylate (Ts), C(=O)—alkyl, C(═O)-Bn, . . . ,
R′″=H, alkyl, Ac.

2. The compound according to claim 1, comprising a C-glycoside of general formula: 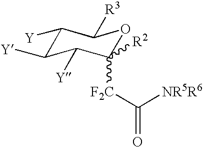
wherein R⁵ and R⁶=H or a group either functionalized or not such as a functionalized carbon chain bearing i.a. an amine, amino acid, aminoester function, a peptide chain, a protein, a carbohydrate, a steroid, or a triterpene, an alkaloid, a lignane, or compounds of pharmacological interest.

3. The compound according to claim 1, comprising a glycoconjugated compound of general formula: 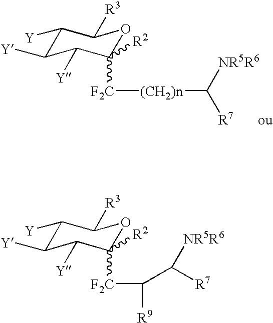
wherein R⁵, R⁶, R⁷ and R⁹=H or a group either functionalized or not, such as a functionalized carbon chain bearing i.a. an amine, amino acid, aminoester function, a peptide chain, a protein, a carbohydrate, a steroid, or a triterpene, an alkaloid, a lignane, or compounds of pharmacological interest.

4. A method for preparing a gem-difluorinated compound of formula: 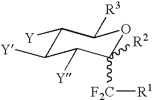
wherein
R¹ is a group comprising an alkyl chain substituted with at least one amine, or amide function,
R² is a hydrogen atom H or a free or protected alcohol function,
R³ is notably an H, CH₃, CH₂OH, CH₂-OGP group wherein GP is a protective group such as an alkyl, benzyl (Bn), trimethylsilyl (TMS), tert-butyl-dimethylsilyl (TBDMS), tert-butyldiphenylsilyl (TBDPS), acetate (Ac) . . . ,
Y, Y′, Y″ are independent groups
wherein Y, Y′, Y″=H, OR, N₃, NR′R″, SR′″ . . .
with R=H, Bn, Ac, TMS, TBDMS, TBDPS, . . . ,
R′, R″=H, alkyl, allyl, Bn, tosylate (Ts), C(=O)-alkyl, C(=O)-Bn, . . . ,
R′″=H, alkyl, Ac,
said method comprising a reaction between a lactone and a halogenated derivative of general formula XCF₂CO₂R⁸, wherein X is a halogen, in the presence of zinc, or of a lanthanide derivative and R⁸=alkyl, aryl . . .

5. The method according to claim 4, wherein said lanthanide derivative is samarium diiodide.

6. The method according to claim 4, wherein said sugar derivative is obtained in one or more steps from a corresponding commercially available sugar.

7. The method according to claim 4, wherein said reaction is followed by a deoxygenation.

8. The method according to claim 4, wherein the R⁸ group comprises an ester function which is reduced to alcohol.

9. The method according to claim 4, wherein the R⁸ group comprises an ester function which is either reduced to alcohol then oxidized into an aldehyde or hemiacetal, or directly reduced into aldehyde.

10. A method for preparing a gem-difluorinated compound of formula: 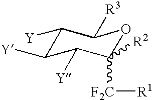
wherein
R¹=C(=O)—NR⁵R⁶, wherein R⁵ and R⁶=H or a group either functionalized or not, such as a functionalized carbon chain bearing i.a. an amine, amino acid, aminoester function, a peptide chain, a protein, a carbohydrate, a steroid, or a triterpene, an alkaloid, a lignane, or compounds of pharmacological interest,
R² is a hydrogen atom H or a free or protected alcohol function,
R³ is an H, CH₃, CH₂OH, CH₂-OGP group wherein GP is a protective group such as an alkyl, benzyl (Bn), trimethylsilyl (TMS), tert-butyl-dimethylsilyl (TBDMS), tert-butyldiphenylsilyl (TBDPS), acetate (Ac) . . . ,
Y, Y′, Y″ are independent groups
wherein Y, Y′, Y″=H, OR, N₃, NR′R″, SR′″ . . .
with R=H, Bn, Ac, TMS, TBDMS, TBDPS, . . . ,
R′, R″=H, alkyl, allyl, Bn, tosylate (Ts), C(=O)-alkyl, C(=O)-Bn, . . . ,
R′″=H, alkyl, Ac,
said method comprising a Ugi reaction with an amine, an aldehyde and an isonitrile.

11. A method for preparing a gem-difluorinated compound of formula: 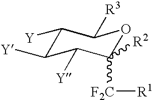
wherein
R¹=—C(=O)-NR⁵R⁶, wherein R⁵ and R⁶=H or a group either functionalized or not, such as a functionalized carbon chain bearing i.a. an amine, amino acid, aminoester function, a peptide chain, a protein, a carbohydrate, a steroid, or a triterpene, an alkaloid, a lignane, or compounds of pharmacological interest,
R² is a hydrogen atom H or a free or protected alcohol function,
R³ is an H, CH₃, CH₂OH, CH₂-OGP group wherein GP is a protective group such as an alkyl, benzyl (Bn), trimethylsilyl (TMS), tert-butyl-dimethylsilyl (TBDMS), tert-butyldiphenylsilyl (TBDPS), acetate (Ac) . . . ,
Y, Y′, Y″ are independent groups
wherein Y, Y′, Y″=H, OR, N₃, NR′R″, SR′″ . . .
with R=H, Bn, Ac, TMS, TBDMS, TBDPS, . . . ,
R′, R″=H, alkyl, allyl, Bn, tosylate (Ts), C(=O)-alkyl, C(=O)-Bn, . . . ,
R′″=H, alkyl, Ac,
said method comprising a coupling reaction of a sugar derivative with an amine.

12. A composition, comprising at least one compound according to claim 1 or one of its derivatives or one of its salts obtained by addition to a pharmaceutically acceptable organic or mineral acid.

13. The use of a gem-difluorinated compound according to claim 1, for preparing antitumoral drugs.

14. The use of a gem-difluorinated compound according to claim 1, for preparing antiviral drugs.

15. The use of a gem-difluorinated compound according to claim 1, for preparing hypoglycemic drugs.

16. The use of a gem-difluorinated compound according to claim 1, for preparing compounds for immunology.

17. The use of a gem-difluorinated compound according to claim 1, for preparing anti-inflammatory compounds.

18. The use of a gem-difluorinated compound according to claim 1, for preparing compounds for cosmetology.

19. The use of a gem-difluorinated compound according to claim 1, for preparing glycopeptide analogs of antifreeze molecules.
